(19) United States
(12) Patent Application Publication
(10) Pub. No.: US 2023/0338305 A1
(43) Pub. Date: Oct. 26, 2023

(54) VITAMIN A FOR USE IN THE TREATMENT OF TRAUMATIC BRAIN INJURY

(71) Applicant: REGENALL LIMITED, London (GB)

(72) Inventor: Mohamed Hamza NOORDEEN, London, Greater London (GB)

(73) Assignee: REGENALL LIMITED, London (GB)

(21) Appl. No.: 17/923,105

(22) PCT Filed: May 6, 2021
(86) PCT No.: PCT/GB2021/051104

§ 371 Date: Nov. 3, 2022

(30) Foreign Application Priority Data
May 6, 2020 (GB) 2006727.8
Apr. 20, 2021 (GB) 2105648.6

(51) Int. Cl. A61P 25/00; A61K 31/07; A61K 31/203

(52) U.S. Cl. CPC A61K 31/07; A61K 31/203; A61P 25/00

(57) ABSTRACT

Vitamin A, and compositions, combined preparations, and multiple-dose formulations comprising vitamin A, for use in the treatment of acute and chronic traumatic brain injury are described, as well as methods of treatment of such injury using vitamin A, or the compositions, combined preparations, or multiple-dose formulations.

This invention relates to compounds, compositions, combined preparations, and multiple-dose formulations, for use in the treatment of acute and chronic traumatic brain injury, and of brain disorders with delayed onset following traumatic brain injury, and to methods of treatment of such injuries and disorders using the compounds, compositions, combined preparations, or multiple-dose formulations.

Traumatic brain injury (TBI) is generally divided into acute TBI and chronic TBI. Acute TBI in sports-related trauma may lead to concussion, subconcussion, haemorrhage or other structural brain damage. Concussion, also known as mild traumatic brain injury (mTBI), is typically defined as a head injury that temporarily affects brain functioning. It may be caused by impact forces, in which the head strikes or is struck by something, or impulsive forces, in which the head moves without itself being subject to blunt trauma. Forces may cause linear, rotational, or angular movement of the brain or a combination of them. The amount of rotational force is thought to be the major component in concussion and its severity. Concussion is the most common form of acute TBI in high-impact sports. Symptoms of concussion may include loss of consciousness, memory loss, headaches, difficulty with thinking, concentration or balance, nausea, blurred vision, sleep disturbances, and mood changes. Any of these symptoms may begin immediately, or appear days after the injury. It is not unusual for symptoms to last two weeks in adults and four weeks in children.

Post-concussion syndrome (PCS) is the presence of persistent neurological symptoms lasting for more than 3 months and is observed in 40-80% of individuals exposed to mild TBI. About 10-15% of individuals experience persistent symptoms after 1 year. Neuropsychological tests reveal that cognitive impairment often persists beyond the subjectively symptomatic time in boxers following mild TBI or a knockout. The seemingly mild head injury causing these subtle subjective and objective neuropsychiatric deficits is sometimes referred to as subconcussion. There is no established treatment for PCS.

Another concussion before the symptoms of a prior concussion have resolved is associated with worse outcomes. Second impact syndrome (SIS) may develop where someone who has sustained an initial head injury, most often a concussion, sustains a second head injury days or weeks after the initial injury, and before its symptoms have fully cleared. The second head injury is typically only a minor blow to the head, but within minutes, the brain swells dangerously and can herniate. The brain stem can fail within five minutes. Except in boxing, all cases have occurred in athletes under the age of 20. Due to the very small number of documented cases, however, the diagnosis is controversial.

Concussion may lead to deleterious effects including reduced brain resistance to a variety of brain disorders with delayed onset. There is a strong positive correlation between concussion and depression, Parkinson’s disease, and anxiety disorders. The severity of concussions and their symptoms may worsen with successive injuries, even if a subsequent injury occurs months or years after an initial one. Repetitive TBI is firmly linked with dementia. Cumulative effects may include psychiatric disorders and loss of long-term memory. For example, the risk of developing clinical depression has been found to be significantly greater for retired American football players with a history of three or more concussions than for those with no concussion history. Three or more concussions is also associated with a fivefold greater chance of developing Alzheimer’s disease earlier and a threefold greater chance of developing memory deficits.

Chronic traumatic encephalopathy (CTE) is a neurodegenerative disease caused by repeated head injuries. The condition was previously referred to as “dementia pugilistica”, or “punch drunk” syndrome, as it was first noted in boxers. Symptoms do not typically begin until years after the injuries. The disease can lead to cognitive and physical handicaps such as parkinsonism, speech and memory problems, slowed mental processing, tremor, depression, and inappropriate behaviour. It shares features with Alzheimer’s disease. Most documented cases have occurred in athletes involved in contact sports such as boxing, American football, professional wrestling, ice hockey, rugby, and soccer. The exact amount of trauma required for the condition to occur is unknown, and definitive diagnosis can currently only occur at autopsy.

CTE is classified as a tauopathy. The neuropathological appearance of CTE is distinguished from other tauopathies, such as Alzheimer’s disease. The macroscopic features of CTE include diffuse brain atrophy, ventricular dilatation, cavum septum pellucidum with or without fenestrations, cerebellar scarring and depigmentation and degeneration of the substantia nigra. Marked atrophy of the medial temporal lobe, thalamus, hypothalamus and mammillary bodies becomes evident in advanced CTE. CTE pathology at the microscopic level includes extensive neurofibrillary tangles (NFTs) composed of mixed 3-repeat (3R) and 4-repeat (4R) tau isoforms. NFTs and astrocytic tangles in CTE are most abundant in the frontal and temporal cortices. Although both are mixed 3R and 4R tauopathies, CTE is distinct from Alzheimer’s disease in the lack of, or relatively little, Aβ deposition especially in younger individuals and in early stages of CTE. Astrocytic tau pathology in CTE is predominantly 4R tau and is more widely distributed than that observed in ageing and Alzheimer’s disease (see Ling et al., “Neurological consequences of traumatic brain injuries in sports”, Molecular and Cellular Neuroscience 66 (2015) 114:122). No cure currently exists for CTE. Treatment is supportive as with other forms of dementia.

Inappropriate management of concussion and subconcussion may put an athlete at risk of developing SIS and/or chronic PCS (CPCS) with persistent neurological symptoms, most commonly, headache, dizziness, impaired attention, poor memory, executive dysfunction, irritability and depression. CPCS is a type of chronic TBI, which is probably distinct from CTE, and the onset of neurological symptoms begins rapidly after the head trauma and persists but rarely progresses.

The response of neural tissue to concussion is not well characterised. It is known that mild trauma to the brain causes biochemical changes resulting in neural dysfunction and structural abnormalities. When subjected to rapid acceleration, deceleration and rotational forces, the brain and all its components, including neurons, glial cells and blood vessels, are stretched, which may disrupt their normal functions.

Mechanical shearing and stretching forces disrupt the cell membrane of nerve cells through “mechanoporation”. This results in potassium efflux into the extracellular space with the subsequent release of excitatory neurotransmitters, leading to sustained depolarization, impaired nerve activity, and potential nerve damage. In an effort to restore ion balance, sodium-potassium ion pumps increase activity, which results in excessive glucose consumption, quickly depleting glucose stores within the cells. Inefficient oxidative metabolism leads to anaerobic metabolism of glucose and increased lactate accumulation. The resultant local acidosis in the brain and increased cell membrane permeability leads to local swelling. After this increase in glucose metabolism, there is a subsequent lower metabolic state which may persist for up to four weeks after injury.

Axonal swellings occur and axons become disconnected at the location of the injury. Axons that span long distances from the cell bodies are particularly susceptible to stretching, which may lead to diffuse axonal injury. It is possible that concussion leads to axonal injury, loss of microvascular integrity and breach of the blood brain barrier, triggering an inflammatory cascade and microglia and astrocyte activation, forming the basis of a mechanistic link with the subsequent development of chronic traumatic encephalopathy (CTE) (Ling et al., “Neurological consequences of traumatic brain injuries in sports”, Molecular and Cellular Neuroscience 66 (2015) 114:122).

Tissue repair encompasses two separate processes: regeneration and replacement. Regeneration refers to a type of healing in which new growth completely restores portions of damaged tissue to their normal state. Replacement refers to a type of healing in which severely damaged or non-regenerable tissues are repaired by the laying down of connective tissue (or glial tissue in the brain), a process commonly referred to as scarring. Tissue repair may restore some of the original structures of the damaged tissue, but may also result in structural abnormalities that impair function.

In the central nervous system (CNS), glial scar grows as a major physical and chemical barrier against regeneration of neurons as it forms dense isolation and creates an inhibitory environment, resulting in limitation of optimal neural function. Glial scar is mainly attributed to the activation of resident astrocytes which surround the lesion core and wall off intact neurons. Glial cells induce the infiltration of immune cells, resulting in transient increase in extracellular matrix deposition and inflammatory factors which inhibit axonal regeneration, impede functional recovery, and may contribute to the occurrence of neurological complications.

Traumatic brain injury causes death of neurons and glia around the site of the injury, shearing of ascending and descending axons, and damage to the vasculature. This leads to haemorrhage at the lesion, and release of factors associated with glial scar formation and immune response. Astrocytes and microglia quickly begin to accumulate around the lesion and increase the expression of pro-inflammatory cytokines and chemokines that inhibit axonal regeneration. Increased levels of pro-inflammatory cytokines, myelin debris, and chondroitin sulphate proteoglycans (CSPGs) in the glial scar contribute to secondary damage to neurons, oligodendrocytes, and dystrophic endings of axonal dieback and inhibit the recovery. Perivascular fibroblasts are attracted by haematogenous macrophages, which infiltrate the lesion, and the perivascular fibroblasts form the fibrotic part of the scar (Wang et al., “Portrait of glial scar in neurological diseases”, International Journal of Immunopathology and Pharmacology, Vol. 31, 1-6, 2018).

Strategies to treat concussion are merely those to alleviate the symptoms by physical and cognitive rest. The rationale for rest is that during the acute post-injury period of increased metabolic demand and limited ATP reserves, non-essential activity draws oxygen and glycogen away from damaged neural tissue and slows regeneration and replacement. Other approaches to treatment of concussion are specific to different clinical subtypes. Paracetamol or NSAIDs may be recommended to help with a headache. For patients who experience vision impairments, specific rehabilitation interventions may be used involving exposures to various stimuli. Physiotherapy may be useful for persistent balance problems; cognitive behavioural therapy may be useful for mood changes. For chronic concussion, pharmacological intervention is normally used. For acute or sub-acute phases after injury, a “wait and see” approach is usually implemented due to the heterologous nature of concussion and hence its individual requirements for treatment.

It is clear that there is no effective treatment for acute and chronic TBI other than attempts to alleviate the obvious symptoms of these conditions. There is a need, therefore, to provide improved treatments for acute and chronic TBI.

The applicant has appreciated that inhibition of glial scar tissue formation following TBI may be a key aspect of the repair process, and in particular is a necessary prerequisite for successful tissue regeneration. The applicant has also recognised that vitamin A may be used to inhibit glial scar tissue formation, and may thus be used in the effective treatment of acute and chronic TBI, and of brain disorders with delayed onset following TBI.

According to the invention there is provided Vitamin A for use in the treatment of acute or chronic traumatic brain injury (TBI) in a subject.

According to the invention there is also provided use of vitamin A in the manufacture of a medicament for the treatment of acute or chronic TBI in a subject.

There is also provided according to the invention a method of treating acute or chronic TBI in a subject, which comprises administering to the subject an effective amount of vitamin A.

Optionally the acute or chronic TBI is concussion.

Optionally the acute or chronic TBI is post-concussion syndrome (PCS).

Optionally the chronic TBI is chronic traumatic encephalopathy (CTE).

Administration of vitamin A to a subject following a TBI may also prevent, treat, or ameliorate a brain disorder with delayed onset following the TBI.

There is also provided according to the invention vitamin A for use in the prevention, treatment, or amelioration of a brain disorder with delayed onset following a TBI in a subject.

There is also provided according to the invention use of vitamin A in the manufacture of a medicament for the prevention, treatment, or amelioration of a brain disorder with delayed onset following a TBI in a subject.

The invention also provides method of preventing, treating, or ameliorating a brain disorder with delayed onset following a TBI in a subject, which comprises administering to the subject an effective amount of vitamin A.

The TBI may be an acute or chronic TBI, such as concussion, PCS, or CTE. Optionally the acute or chronic TBI is concussion. Optionally the acute or chronic TBI is PCS.

Optionally the brain disorder with delayed onset is CTE, depression, Parkinson’s disease, dementia, or an anxiety disorder.

In particular, vitamin A is able to treat acute or chronic TBI, or to prevent, treat, or ameliorate a brain disorder with delayed onset following a TBI, by inhibiting formation of glial scar tissue in the brain of the subject following the TBI.

Optionally the TBI was sustained by the subject during participation in a sport.

Optionally the subject is an athlete, or was an athlete when the TBI was sustained.

Mild TBI (concussion) is a relatively common occurrence in several sports, especially contact sports, such as boxing, American football, rugby, soccer, baseball, softball, basketball, as well as other sports, including cycling, water sports, winter sports, horse riding, hockey, ball sports, skating (see Table 2 of Ling et al (supra) for a list of top 20 sports and recreational activities with the highest risk of head injuries requiring hospital emergency care or evaluation). Optionally the TBI was sustained by the subject during participation in a contact sport.

Vitamin A is the name of a group of fat-soluble retinoids, including retinol, retinal, and retinyl esters. There are two different categories of vitamin A. The first category, preformed vitamin A, comprises retinol and its esterified form, retinyl ester. The second category, provitamin A, comprises provitamin A carotenoids such as alpha-carotene, beta-carotene and beta-cryptoxanthin. Both retinyl esters and provitamin A carotenoids are converted to retinol, which is oxidized to retinal and then to retinoic acid. Both provitamin A and preformed vitamin A are known be metabolized intracellularly to retinal and retinoic acid, the bioactive forms of vitamin A.

Vitamin A for use according to the invention may be an isolated form of vitamin A. An isolated form of vitamin A is any form of vitamin A found in the diet or a metabolized form thereof. For example, vitamin A may be isolated from fish liver oil. Vitamin A may comprise a preformed vitamin A such as retinol or a retinyl ester. Retinyl esters include retinyl acetate and retinyl palmitate. Vitamin A may comprise a provitamin A, such as a provitamin A carotenoid including alpha-carotene, beta-carotene or beta-cryptoxanthin. Vitamin A may comprise a bioactive form of vitamin A such as retinal or retinoic acid.

Vitamin A is available for human consumption in multivitamins and as a stand-alone supplement, often in the form of retinyl acetate or retinyl palmitate. A portion of the vitamin A in some supplements is in the form of beta-carotene and the remainder is preformed vitamin A; others contain only preformed vitamin A or only beta-carotene. Supplement labels usually indicate the percentage of each form of the vitamin. The amounts of vitamin A in stand-alone supplements range widely. Multivitamin supplements typically contain 2,500 to 10,000 international units (IU) vitamin A, often in the form of both retinol and beta-carotene.

Vitamin A is listed on food and supplement labels in international units (IUs). However, Recommended Dietary Allowance (RDA) (average daily level of intake sufficient to meet the nutrient requirements of nearly all (97%-98%) healthy individuals) for vitamin A is given as micrograms (µg; mcg) of retinol activity equivalents (RAE) to account for the different bioactivities of retinol and provitamin A carotenoids (see Table 1). Because the body converts all dietary sources of vitamin A into retinol, 1 mcg of physiologically available retinol is equivalent to the following amounts from dietary sources: 1 mcg of retinol, 12 mcg of beta-carotene, and 24 mcg of alpha-carotene or beta-cryptoxanthin. From dietary supplements, the body converts 2 mcg of beta-carotene to 1 mcg of retinol.

Conversion rates between mcg RAE and IU are as follows:
1 IU retinol = 0.3 mcg RAE;
1 IU beta-carotene from dietary supplements = 0.15 mcg RAE;
1 IU beta-carotene from food = 0.05 mcg RAE; and
1 IU alpha-carotene or beta-cryptoxanthin = 0.025 mcg RAE.

An RAE cannot be directly converted into an IU without knowing the source(s) of vitamin A. For example, the RDA of 900 mcg RAE for adolescent and adult men is equivalent to 3,000 IU if the food or supplement source is preformed vitamin A (retinol). However, this RDA is also equivalent to 6,000 IU of beta-carotene from supplements, 18,000 IU of beta-carotene from food, or 36,000 IU of alpha-carotene or beta-cryptoxanthin from food. So a mixed diet containing 900 mcg RAE provides between 3,000 and 36,000 IU of vitamin A, depending on the foods consumed.

TABLE 1

Recommended Dietary Allowances (RDAs) for Vitamin A

| | | | | |
|---|---|---|---|---|
| Age | Male | Female | Pregnancy | Lactation |
| 0-6 months* | 400 mcg RAE | 400 mcg RAE | | |
| 7-12 months* | 500 mcg RAE | 500 mcg RAE | | |
| 1-3 years | 300 mcg RAE | 300 mcg RAE | | |
| 4-8 years | 400 mcg RAE | 400 mcg RAE | | |
| 9-13 years | 600 mcg RAE | 600 mcg RAE | | |
| 14-18 years | 900 mcg RAE | 700 mcg RAE | 750 mcg RAE | 1,200 mcg RAE |
| 19-50 years | 900 mcg RAE | 700 mcg RAE | 770 mcg RAE | 1,300 mcg RAE |
| 51 + years | 900 mcg RAE | 700 mcg RAE | | |

* Adequate Intake (AI), equivalent to the mean intake of vitamin A in healthy, breastfed infants.
Source: National Institutes of Health, Vitamin A, Fact Sheet for Health Professionals, as updated 5 Oct. 2018

The Food and Nutrition Board (FNB) at the Institute of Medicine of the National Academies (formerly National Academy of Sciences) has established tolerable Upper Intake Level (UL) (maximum daily intake unlikely to cause adverse health effects) for preformed vitamin A that apply to both food and supplement intakes. The FNB based these ULs on the amounts associated with an increased risk of liver abnormalities in men and women, teratogenic effects, and a range of toxic effects in infants and children. The FNB has not established ULs for beta-carotene and other provitamin A carotenoids.

TABLE 2

Tolerable Upper Intake Levels (ULs) for Preformed Vitamin A*

| | | | | |
|---|---|---|---|---|
| Age | Male | Female | Pregnancy | Lactation |
| 0-12 months | 600 mcg RAE (2,000 IU): | 600 mcg RAE (2,000 IU) | | |
| 1-3 years | 600 mcg RAE (2,000 IU) | 600 mcg RAE (2,000 IU) | | |
| 4-8 years | 900 mcg RAE (3,000 IU) | 900 mcg RAE (3,000 IU) | | |
| 9-13 years | 1,700 mcg RAE (5,667 IU) | 1,700 mcg RAE (5,667 IU) | | |
| 14-18 years | 2,800 mcg RAE (9,333 IU) | 2,800 mcg RAE (9,333 IU) | 2,800 mcg RAE (9,333 IU) | 2,800 mcg RAE (9,333 IU) |
| 19+ years | 3,000 mcg RAE (10,000 IU) | 3,000 mcg RAE (10,000 IU) | 3,000 mcg RAE (10,000 IU) | 3,000 mcg RAE (10,000 IU) |

Source: National Institutes of Health, Vitamin A, Fact Sheet for Health Professionals, as updated 5 Oct. 2018

* These ULs, expressed in mcg and in IUs (where 1 mcg = 3.33 IU), only apply to products from animal sources and supplements whose vitamin A comes entirely from retinol or ester forms, such as retinyl palmitate. However, many dietary supplements (such as multivitamins) do not provide all of their vitamin A as retinol or its ester forms. For example, the vitamin A in some supplements consists partly or entirely of beta-carotene or other provitamin A carotenoids. In such cases, the percentage of retinol or retinyl ester in the supplement should be used to determine whether an individual’s vitamin A intake exceeds the UL. For example, a supplement labeled as containing 10,000 IU of vitamin A with 60% from beta-carotene (and therefore 40% from retinol or retinyl ester) provides 4,000 IU of preformed vitamin A. That amount is above the UL for children from birth to 13 years but below the UL for adolescents and adults.

It will be appreciated that use of a natural vitamin for treatment of acute or chronic TBI, or for prevention, treatment, or amelioration of a brain disorder with delayed onset following a TBI, is particularly advantageous because of its known safety profile.

Preferably vitamin A for use according to the invention, or use of vitamin A according to the invention, comprises a high dose of vitamin A.

A high dose of vitamin A is considered to be a dose that exceeds a UL for the subject. Examples of high doses of vitamin A include: >10,000 IU to 100,000 IU vitamin A per day; about 25,000 to 50,000 IU vitamin A per day; about 25,000 to 75,000 IU vitamin A per day; about 25,000 to 100,000 IU vitamin A per day; about 50,000 to 100,000 IU vitamin A per day; or about 75,000 to 100,000 IU vitamin A per day, in particular of preformed vitamin A.

Vitamin A may be administered to the subject once per day, twice per day, three times per day, four times per day, or five times per day.

Vitamin A may be administered to the subject for at least 3 days for example for at least a week, for at least a month, or for at least 6 months from the day of first administration to the subject.

Prolonged exposure to high doses of vitamin A may lead to hypervitaminosis A. Thus, it may be preferred to limit administration of high doses of vitamin A to the subject for up to 6 years, or up to 6 months, from the day of first administration to the subject.

Optionally for an adult human subject (>18 years old), the subject may be administered up to 100,000 IU vitamin A (in particular of preformed vitamin A) per day for up to 6 months. For example, >10,000 IU to 100,000 IU vitamin A per day; about 25,000 to 100,000 IU vitamin A per day; about 50,000 to 100,000 IU vitamin A per day; or about 75,000 to 100,000 IU vitamin A per day (in particular of preformed vitamin A) for up to 6 months.

Optionally for an adult human subject (>18 years old), the subject may be administered up to 25,000 IU vitamin A per day (in particular of preformed vitamin A) for up to 6 years. For example, >10,000 IU to 25,000 IU vitamin A per day (in particular of preformed vitamin A) for up to 6 years.

Ongoing administration (for example, ongoing daily administration) of vitamin A for weeks, months, or years, to the subject may be particularly effective in preventing, or reducing the risk of, the subject developing a brain disorder with delayed onset, such as Parkinson’s disease, CTE, depression, an anxiety disorder, or dementia.

Optionally the subject is administered up to 50% (for example >10% to 50%, or 25% to 50%) of a maximum safe dose of vitamin A (in particular of preformed vitamin A) for the subject per day.

For example, a maximum safe dose of vitamin A (in particular of preformed vitamin A) per day for an adult human subject may be 100,000 IU vitamin A (in particular of preformed vitamin A).

The vitamin A may be administered to a subject systemically, for example, orally or intravenously.

Without being bound by theory, it is believed that inhibition of glial scar tissue formation in accordance with the invention facilitates regeneration of normal tissue, in particular by stem cells and/or quiescent cells present within or near damaged tissue. Quiescence is the reversible state of a cell in which it does not divide but retains the ability to re-enter cell proliferation. Some adult stem cells are maintained in a quiescent state and can be rapidly activated when stimulated, for example by damage or injury to the tissue in which they reside.

It will be appreciated that vitamin A should preferably be administered as soon as possible after a traumatic brain injury has occurred. Optionally vitamin A is administered within a month, within a week, within a day, within a few hours (for example, within 12 or 6 hours), or within an hour of the traumatic brain injury causing concussion.

However, beneficial effects of treatment with vitamin A may also be observed when treatment is initiated many weeks, months, or even years after an injury has occurred. Thus, optionally vitamin A may be administered weeks, months, years or even decades after an injury has occurred, for example within six weeks, six months, a year, or a decade, or within twenty, thirty, forty, fifty, or sixty years of the injury.

Optionally the subject is not vitamin A deficient.

Plasma retinol levels are typically measured to assess vitamin A status. However, plasma retinol levels are under tight hepatic homeostatic control and do not decline until vitamin A concentration in the liver is almost depleted (critical liver concentration ≤20 µg g⁻¹ of liver). Liver vitamin A reserves can be measured indirectly through the relative dose-response test (McLaren, D.S.; Kraemer, K. Manual on Vitamin Deficiency Disorders (VADD), 3rd ed.; Sight and Life Press:Basel, Switzerland, 2012; ISBN 978-3-906412-58-0), which is considered the “gold standard” indicator of whole-body vitamin A status. However, for clinical purposes, plasma retinol levels alone are sufficient and commonly used for documenting significant deficiency of vitamin A. The physiological plasma concentration of vitamin A is 1-2 µmol/L and, according to the World Health Organization, values of serum retinol concentrations below a cut-off of 0.70 µmol/L (or 20 µg/dL) represent biochemical vitamin A deficiency (VAD), and values lower than 0.35 µmol/L are indicative of severe deficiency and associated with numerous clinical manifestations.

Optionally the subject has a serum retinol concentration of at least 0.7 µmol/L.

Optionally the subject has a plasma concentration of vitamin A of 1-2 µmol/L.

Optionally the vitamin A is to be administered to the subject at a dose that results in a plasma concentration of vitamin A in excess of 2 µmol/L.

Also provided according to the invention is a multiple-dose formulation comprising a plurality of separate unit doses of vitamin A wherein each unit dose comprises up to 100,000 IU vitamin A, for example >10,000 IU to 100,000 IU vitamin A; 25,000 to 50,000 IU vitamin A, 25,000 to 75,000 IU vitamin A, 25,000 to 100,000 IU vitamin A; 50,000 to 100,000 IU vitamin A; or 75,000 to 100,000 IU vitamin A (in particular of preformed vitamin A).

Vitamin A of a multiple-dose formulation of the invention may comprise any combination of vitamin A described previously.

A multiple-dose formulation of the invention may comprise at least 7 unit doses, at least 30 unit doses, or at least 100 unit doses of vitamin A.

Each unit dose of vitamin A in a multiple-dose formulation of the invention may comprise a pharmaceutical composition comprising vitamin A and a pharmaceutically acceptable carrier, excipient or diluent.

There is also provided according to the invention a multiple-dose formulation for use in inhibition of glial scar tissue formation in a subject.

Use of vitamin A in accordance with the invention may be particularly effective for the treatment of older subjects. For example, a human subject may be at least 18 years old, at least 25 years old, at least 30 years old, at least 40 years old, or at least 50 years old.

Diagnosis of concussion may be based on physical and neurological examination findings, duration of unconsciousness (usually less than 30 minutes) and post-traumatic amnesia (PTA; usually less than 24 hours), and the Glasgow Coma Scale (MTBI sufferers have scores of 13 to 15) (Borg et al., “Diagnostic procedures in mild traumatic brain injury: results of the WHO Collaborating Centre Task Force on Mild Traumatic Brain Injury”, Journal of Rehabilitation Medicine, 36 (43 Suppl): 61-75, 2004). Neuropsychological tests exist to measure cognitive function (Moser, et al., “Neuropsychological evaluation in the diagnosis and management of sports-related concussion”, Archives of Clinical Neuropsychology, 22 (8): 909-16, 2007). Such tests may be administered hours, days, or weeks after the injury, or at different times to demonstrate any trend. Increasingly, athletes are also being tested pre-season to provide a baseline for comparison in the event of an injury, though this may not reduce risk or affect return to play.

A diagnosis of PCS may be made when symptoms resulting from concussion last for more than three months after the injury. The International Statistical Classification of Diseases and Related Health Problems (ICD-10) sets out criteria for post-concussion syndrome (PCS) (Boake et al. (2005). “Diagnostic criteria for postconcussional syndrome after mild to moderate traumatic brain injury”. Journal of Neuropsychiatry and Clinical Neurosciences. 17 (3): 350-6.). To meet the ICD-10 criteria, a patient has had a head injury “usually sufficiently severe to result in loss of consciousness” and then develops at least three of the following eight symptoms within four weeks: headache izziness, atigue, rritability, sleep problems, concentration problems, memory problems, problems tolerating stress/emotion/alcohol. Neuropsychological tests exist to measure deficits in cognitive functioning that can result from PCS (Hall et al. (2005). “Definition, diagnosis, and forensic implications of postconcussional syndrome”. Psychosomatics. 46 (3): 195-202). The Stroop Color Test and the 2&7 Processing Speed Test (which both detect deficits in speed of mental processing) can predict the development of cognitive problems from PCS. A test called the Rivermead Postconcussion Symptoms Questionnaire, a set of questions that measure the severity of 16 different post-concussion symptoms, can be self-administered or administered by an interviewer (Mittenberg and Strauman (2000). “Diagnosis of mild head injury and the postconcussion syndrome”. Journal of Head Trauma Rehabilitation. 15 (2): 783-791). Other tests that can predict the development of PCS include the Hopkins Verbal Learning A test (HVLA) and the Digit Span Forward examination.

Corsellis et al. (“The aftermath of boxing” (1973) Psychol. Med. 3, 270-303) proposed four major criteria for diagnosis of CTE: 1. Abnormalities of the septum pellucidum (i.e., cavum, fenestrations), 2. Cerebellar scarring on the inferior surface of the lateral lobes (especially the tonsillar regions), 3. Degeneration of the substantia nigra (pallor) and 4. Widespread NFTs containing hyperphosphorylated tau in the cerebral cortex and brainstem. Two recent neuropathological criteria have since been proposed (McKee et al., 2013. “The spectrum of disease in chronic traumatic encephalopathy”. Brain 136, 43-64; Omalu et al., 2011. “Emerging histomorphologic phenotypes of chronic traumatic encephalopathy in American athletes”. Neurosurgery 69, 173-183). Omalu et al. identified four phenotypes of CTE and McKee et al. classified CTE into four pathological stages.

The effects of head trauma may be seen with the use of structural imaging. Imaging techniques include the use of magnetic resonance imaging, nuclear magnetic resonance spectroscopy, CT scan, single-photon emission computed tomography, Diffusion MRI, and Positron Emission Tomography (PET). A PET scan may also be used to evaluate tau deposition.

The term “treatment” is used herein to include a prevention or lessening of any of the symptoms of an acute or chronic TBI, such as concussion, PCS, or CTE.

Symptoms of concussion include loss of consciousness, memory loss, headaches, difficulty with thinking, concentration or balance, nausea, blurred vision, sleep disturbances, and mood changes.

Symptoms of PCS include persistent neurological symptoms, most commonly, headache, dizziness, impaired attention, poor memory, executive dysfunction, irritability depression, noise sensitivity, and anxiety.

Symptoms of CTE include unsteadiness of gait, mental confusion, slowing of muscular movements, and, occasionally, hesitancy in speech, tremors of the hands and nodding of the head. Behavioural disturbances are usually the earliest findings in CTE and may include depression, mood swings, apathy, impulsivity, aggression and suicidality. Cognitive deficits include attention and concentration impairment, memory problems, executive dysfunction and eventually dementia. Common motor symptoms are parkinsonism, tremor, dysarthria, coordination difficulties and ataxia, reflect extrapyramidal and pyramidal system and cerebellum involvements. Headache is another prominent feature but may represent comorbid CPCS (Ling et al., 2015, supra). In 2014, a large cohort of pathologically confirmed CTE delineated CTE into two clinical phenotypic presentations: one with predominant mood and behavioural symptoms in younger individuals in the third decade and another with cognitive impairment presenting in the fifth decade (Stern et al., 2013. “Clinical presentation of chronic traumatic encephalopathy”. Neurology 81, 1122-1129).

Symptoms of acute or chronic TBI, such as concussion or PCS, also include reduced brain resistance to a variety of brain disorders with delayed onset, such as CTE, depression, Parkinson’s disease, dementia, and anxiety disorders. Repeated concussions may also increase the risk in later life of chronic traumatic encephalopathy (CTE), Parkinson’s disease, dementia, anxiety disorders, and depression.

The vitamin A can be incorporated into a variety of formulations for therapeutic administration, more particularly by combination with appropriate, pharmaceutically acceptable carriers, pharmaceutically acceptable diluents, or other pharmaceutically acceptable excipients, and can be formulated into preparations in solid, semi-solid, liquid or gaseous forms, such as tablets, capsules, powders, granules, ointments, solutions, suppositories, injections, inhalants and aerosols as appropriate.

Optionally the vitamin A is in solid form.

Optionally the vitamin A is not in an organic solution.

Optionally the vitamin A is not encapsulated by, or attached to a microparticle.

Optionally the vitamin A is not encapsulated by, or attached to a nanoparticle.

Vitamin A can be administered in the form of a pharmaceutically acceptable salt. It can also be used alone or in appropriate association, as well as in combination, with other pharmaceutically active compounds. Optionally vitamin A is administered with an antibiotic agent. Optionally vitamin A is the only non-cellular, non-antibiotic, active agent administered.

The following methods and excipients are merely exemplary and are in no way limiting.

For oral preparations, vitamin A can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Vitamin A can be formulated into preparations for injection by dissolving, suspending or emulsifying in an aqueous or nonaqueous solvent, such as vegetable or other similar oils, propylene glycol, synthetic aliphatic acid glycerides, injectable organic esters (e.g., ethyl oleate), esters of higher aliphatic acids or propylene glycol; and if desired, with conventional additives such as solubilizers, isotonic agents, suspending agents, emulsifying agents, stabilizers and preservatives. Parenteral vehicles include sodium chloride solution, Ringer’s dextrose, dextrose and sodium chloride, lactated Ringer’s, or fixed oils. Intravenous vehicles include fluid and nutrient replenishers, electrolyte replenishers (such as those based on Ringer’s dextrose), and the like. Furthermore, a pharmaceutical composition of the present disclosure can comprise further agents such as dopamine or psychopharmacologic drugs, depending on the intended use of the pharmaceutical composition.

Pharmaceutical compositions are prepared by mixing Vitamin A having the desired degree of purity, with optional physiologically acceptable carriers, other excipients, stabilizers, surfactants, buffers and/or tonicity agents. Acceptable carriers, other excipients and/or stabilizers are nontoxic to recipients at the dosages and concentrations employed, and may include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition can be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents can be used for the production of pharmaceutical compositions for parenteral administration; see also Chen (1992) Drug Dev Ind Pharm 18, 1311-54.

An aqueous formulation can be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent can be included in the formulation to modulate the tonicity of the formulation. Exemplary tonicity agents include sodium chloride, potassium chloride, glycerin and any component from the group of amino acids, sugars as well as combinations thereof. In some embodiments, the aqueous formulation is isotonic, although hypertonic or hypotonic solutions can be suitable. The term “isotonic” denotes a solution having the same tonicity as some other solution with which it is compared, such as a physiological salt solution or serum. Tonicity agents can be used in an amount of about 5 mM to about 350 mM, e.g., in an amount of 100 mM to 350 nM.

A surfactant can also be added to the formulation to reduce aggregation and/or minimize the formation of particulates in the formulation and/or reduce adsorption. Exemplary surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Exemplary concentrations of surfactant can range from about 0.001% to about 1% w/v.

A lyoprotectant can also be added in order to protect a labile active ingredient against destabilizing conditions during the lyophilization process. For example, known lyoprotectants include sugars (including glucose and sucrose); polyols (including mannitol, sorbitol and glycerol); and amino acids (including alanine, glycine and glutamic acid). Lyoprotectants can be included in an amount of about 10 mM to 500 nM.

In some embodiments, a subject formulation includes one or more of the above-identified agents (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

Unit dosage forms for oral administration such as syrups, elixirs, and suspensions can be provided wherein each dosage unit, for example, teaspoonful, tablespoonful, or tablet contains a predetermined amount of the active agent (i.e. vitamin A). Similarly, unit dosage forms for injection or intravenous administration can comprise vitamin A in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of vitamin A, calculated in an amount sufficient to produce the desired effect in association with a pharmaceutically acceptable diluent, carrier or vehicle.

Vitamin A can be administered as an injectable formulation. Typically, injectable compositions are prepared as liquid solutions or suspensions; solid forms suitable for solution in, or suspension in, liquid vehicles prior to injection can also be prepared.

Suitable excipient vehicles are, for example, water, saline, dextrose, glycerol, ethanol, or the like, and combinations thereof. In addition, if desired, the vehicle can contain minor amounts of auxiliary substances such as wetting or emulsifying agents or pH buffering agents. Actual methods of preparing such dosage forms are known, or will be apparent, to those skilled in the art. See, e.g., Remington’s Pharmaceutical Sciences, Mack Publishing Company, Easton, Pennsylvania, 17th edition, 1985. The composition or formulation to be administered will, in any event, contain a quantity of vitamin A adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Ranges may be expressed herein as from “about” one particular value, and/or to another particular value. When such a range is expressed, another embodiment includes from the one particular value and/or to the other particular value. Similarly, when values are expressed as approximations, by the use of the antecedent “about”, it will be understood that the particular value forms another embodiment.

Wherever the term “vitamin A” is used herein this includes reference to “vitamin A or a pharmaceutically acceptable salt thereof”.

Methods for visualising glial scar tissue formation are known to those of ordinary skill in the art. Examples include magnetic resonance imaging (MRI). Suitable examples are described in the following documents:
MRI in CNS injury:
   Ellingson et al., “Imaging Techniques in Spinal Cord Injury”, World Neurosurg. 2014 Dec; 82(6): 1351-1358;
   Hu et al., “Glial scar and neuroregeneration: histological, functional, and magnetic resonance imaging analysis in chronic spinal cord injury”, Journal of Neurosurgery, 2010, 13(2) (doi.org/10.3171/2010.3.SPINE09190);
   Byrnes et al., “Neuropathological Differences Between Rats and Mice after Spinal Cord Injury”, J Magn Reson Imaging. 2010 Oct; 32(4): 836-846.

Embodiments of the invention are described below, by way of example only, with reference to the accompanying drawings, in which:

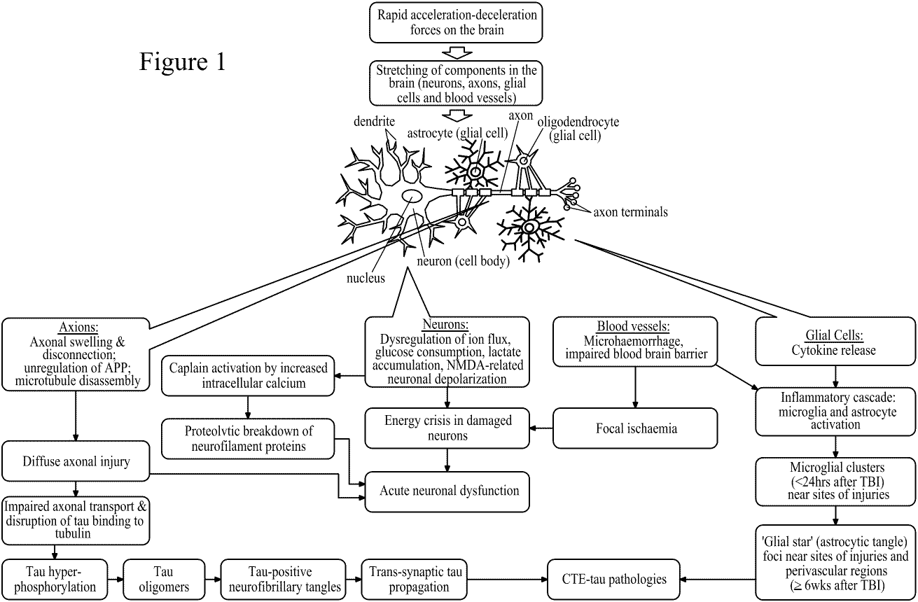

FIG. 1 shows a schematic illustration of the proposed cascade of events triggered by acute TBIs and its possible mechanistic links with the development of CTE pathology (from Ling et al., “Neurological consequences of traumatic brain injuries in sports”, Molecular and Cellular Neuroscience 66 (2015) 114:122);

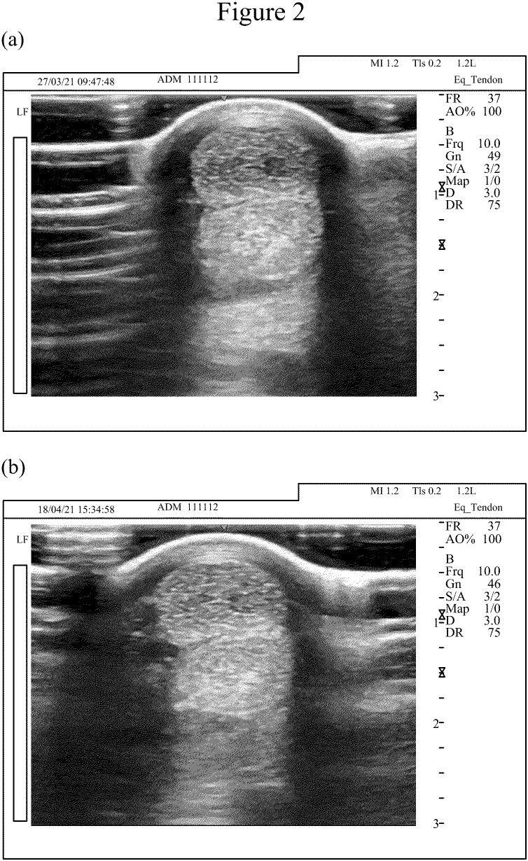

FIG. 2 shows an ultrasonograph of a subtle core lesion in the lateral aspect of superficial digital flexor tendon (SDFT) with generalised surrounding tendonitis of a horse (ID 111112): (a) before administration of any pharmaceutical composition comprising vitamin A; and (b) after daily administration of a pharmaceutical composition comprising vitamin A for 14 days; and

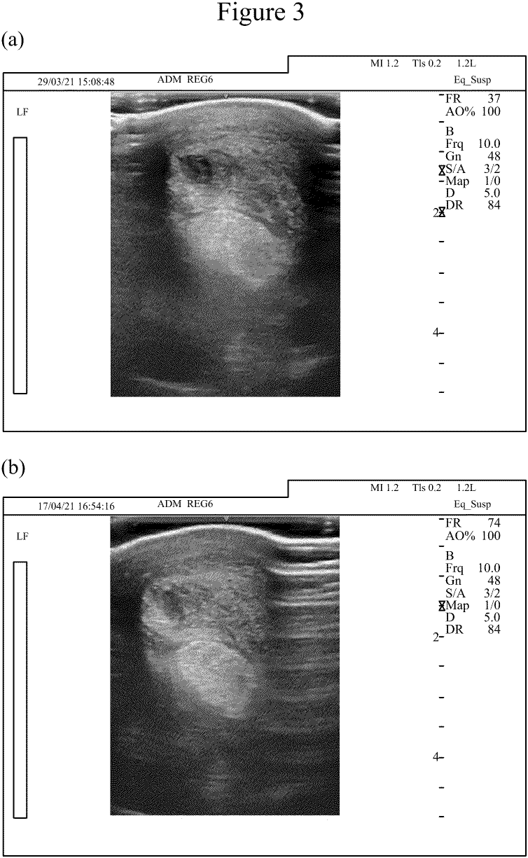

FIG. 3 shows an ultrasonograph of a nasty SDFT core lesion in the medial aspect not quite involving paratenon for a horse (ID REG6): (a) before administration of any pharmaceutical composition comprising vitamin A; and (b) after daily administration of a pharmaceutical composition comprising vitamin A for 14 days.

The following Examples 1-4 describe patient-reported outcome measures (PROMs) for four human patients with spinal cord injury, and their recovery following treatment with vitamin A. Whist not directed to treatment of concussion, these examples demonstrate that vitamin A is effective in treatment of traumatic CNS injury (believed to be as a result of inhibition of glial scar tissue formation in the spinal cord).

### EXAMPLE 1: PATIENT-REPORTED OUTCOME MEASURE

### Spinal Cord Injury - Recovery Following Treatment With Vitamin A

| | |
|---|---|
| Patient | Human Subject 1 |
| Age | 68 |
| Sex | Male |
| Health status | Good |

### Injury

| | | |
|---|---|---|
| Type of injury | Spinal cord injury | |
| When the injury occurred | November, 1963 | |
| Cause of the injury | Trauma to T4 | |
| Tissue(s) affected | Left lateral spinothalamic tract (Brown-Sequard syndrome) | |
| Impairments resulting from the injury | | Left stiff knee, right lower back pain, difficulty walking |

### Treatment With Conventional Methods

Laminectomy T4 and T5

Baclofen 20 mg AM and 30 mg PM for the past 13 years.

### Extent of Recovery Following Treatment With Conventional Methods

Seven years after my operation (1963) I was very active and had a Brown Belt in Tae Kwon Do, I started to limp when I was 24 years old. At 38 years I could not run. At 52 my limp was more pronounced and I had a series of Botox injections to my left leg and thigh muscles over a period of 2-3 years. At 55 I was started on Baclofen and currently I take 20 mg every morning and 30 mg at night. At 64 my mobility continues to decrease and I began using a cane to ambulate. I began to experience pain of my right lower back approximately 4 years ago. It ranges from a scale of 2-6/10 depending on activities I do as well as the weather.

### Treatment With Vitamin A

How soon after the injury was treatment was treatment with Vitamin A initiated?

56 years

Type of Vitamin A administered

Vitamin A 50,000 IU (from Retinyl palmitate and fish liver oil)

Route of administration

Oral

Dose of Vitamin A per administration

50,000 IU

Frequency of administration

Once daily

Period of administration

3 months

### Recovery Following Treatment With Vitamin A

The pain is approximately 10-15% less and when I have it, the duration is much less. The weather conditions have to be severe before I begin to experience pain. Previously I could forecast if it was going to rain a day or two ahead because my right lower back would start to ache. Now the weather conditions have to be more severe for my back to start aching.

### EXAMPLE 2: PATIENT-REPORTED OUTCOME MEASURE

### Spinal Cord Injury - Recovery Following Treatment With Vitamin A

| | |
|---|---|
| Patient | Human Subject 2 |
| Age | 69 |
| Sex | Male |

### Injury

| | |
|---|---|
| Type of injury | Spinal cord injury |
| When the injury occurred | Beginning of September 2016 |
| Cause of the injury | Spinal stenosis |
| Tissue(s) affected | Paralyzed both legs and no feeling up to chest |
| Impairments resulting from the injury | Both legs paralyzed and up to the chest |

### Treatment With Conventional Methods

First surgery 22^{nd} of September 2016, T3 to T4 (Considerable bleeding occurred during the operation). Second surgery in two places (laminotoimiu) on 9^{th} of March 2017, Th10-Th12 and L1-L5.

Drugs: After surgery 2016: Paracetamol 1gx4, Codein 30 mgx4, Valsartan 82,5×1, Atorvastatin 40 mgx1. Omeprazol 20 mgx1, D3 vitamin 2000 aex1, Kaleorid 750 mgx1, Klexane 40 mgx1, Betolvex 1 mgx1, Valsartan/hydrochlortiazide 80/12,5 ×1, Atacor 40 mgx1, Gabapentin 300plus 900 mg, Zopiclone 7,5 mg vesp. Tradolan 50 mg 2×2,

2019: Losartan/Hydrochlorothiazide 100 mg/25 mg x1, Acetylaslisylsyre 75 mgx1, Diclomex Rapid 50 mg 1×3, Anti Leg Cramps (NAVEH) 2×1, Vitamin A10.000×5. Imovane 7,5 mg 1×1. Milk Thistle(Lamberts) 8500 mg of seed-Providing Silymarin 200 mg 1×1.

### Extent of Recovery Following Treatment With Conventional Methods

Daily exercises from the beginning. Wheelchair and crutches. Cramps in legs, need to take Gapabentin 3-5 pr day and Tradolan 2 pr day.

### Treatment With Vitamin A

How soon after the injury was treatment was treatment with Vitamin A initiated?

Starting in October 2018. For three months taking vitamin A 10,000 × 3. Increasing in end January 2019 10,000 × 5.

Type of Vitamin A administered

Vitamin A (from cod liver oil and vitamin A palmitate)

Route of administration

Oral

Dose of Vitamin A per administration

Initially total of 30,000 IU; From end of January 2019 total of 50,000 IU; 3,000 mcg RAE

Frequency of administration

For 3 months 3 tablets a day and for 3 months 5 tablets a day

Period of administration

6 months

### Recovery Following Treatment With Vitamin A

First I found recovery more walking control and balance. Coordination was better. In mid-January 2019 a big pain in left leg and ankle and could not train my left legs. My doctor said it was probably because of one of the pockets with water drops managed to get into my spinal cord. I asked him if that could happen again, he said no, may be only 10% possibility. This problem took me 1.5 months to recover. Now in March 2019 I feel much stronger, more balanced, more movement control and I am walking as a whole 6×42 steps (up and down) twice in a week. In beginning of April start walking without any support on even level 30-50 meters with better controlling.

Subject provided videos showing the subject walking along an even level with very little to no support and walking up and down a set of stairs.

### EXAMPLE 3: PATIENT-REPORTED OUTCOME MEASURE

### Spinal Cord Injury - Recovery Following Treatment With Vitamin A

| | |
|---|---|
| Patient | Human Subject 3 |
| Age | 79 |
| Sex | Male |
| Health status | Good |

### Injury

| | |
|---|---|
| Type of injury | Disc/nerve pain |
| When the injury occurred | 2018 |
| Tissue(s) affected | Facet joint |
| Impairments resulting from the injury | Pain around knee (limping) |

### Treatment With Conventional Methods

Injections to disc/nerve x 2

### Treatment With Vitamin A

How soon after the injury was treatment with Vitamin A initiated?

¾ months

Type of Vitamin A administered

Vitamin A 50,000 IU

Route of administration

Oral

Dose of Vitamin A per administration

50,000:1

Frequency of administration

Daily

Period of administration

4-5 months

### Recovery Following Treatment With Vitamin A

Vitamin A has improved pain level which has almost disappeared x 80%.

### EXAMPLE 4: PATIENT-REPORTED OUTCOME MEASURE

### Spinal Cord Injury - Recovery Following Treatment With Vitamin A

| | |
|---|---|
| Patient | Human Subject 4 |
| Age | 60 |
| Sex | Male |

### Injury

| | | |
|---|---|---|
| Type of injury | Spinal cord injury and now have lumbar spinal stenosis as well | |
| When the injury occurred Cause of the injury Tissue(s) affected | Spinal Cord Injury in March 1998 at C4/C5 level Fell off mountain in France skiing Incomplete injury, initial paralysis below C4/C5 but reasonable mobility has been recovered. All tissues effected below C4/C5 | |
| Impairments resulting from the injury | | Loss of some mobility below C4/C5 level, I walk with a stick. |

### Treatment With Conventional Methods

In 1998 I had a laminectomy after the accident in France, and then a disc fusion in London both at C4/C5 level.

In 2009 I had a further disc replacement in my neck after a fall.

I had injections in my neck in May 2018 for stenosis and severe neck pain.

I have had injections in my lumbar region in May and September 2018 and March 2019 and rhizolysis in the lumbar region in September 2018 and at 3 levels in March 2019.

I have intermittently taken pain killers including ibuprofen, paracetomol and naproxen. In February 2019 I took Arcoxia for 4 weeks. I also take antibiotics as a prophylaxis to prevent UTIs. I also take toltoredine, topsium chloridfe, senna and diactyl.

### Extent of Recovery Following Treatment With Conventional Methods

Good recovery from the original spinal cord injury in my neck but it left me with very impaired mobility and walking with a stick.

After the injection in my neck in May 2018 this significantly helped remove the pain but it left me with a very stiff neck which still ached quite a lot but the pain was bearable.

Improved mobility since the injection and rhizolysis in lumbar region in March 2019.

### Treatment With Vitamin A

How soon after the injury was treatment was treatment with Vitamin A initiated?

21 years later in February 2019

Type of Vitamin A administered

Vitamin A (from cod liver oil and vitamin A palmitate)

Route of administration

Oral

Dose of Vitamin A per administration

Total of 50,000 IU (5 × 10,000 IU tablets)

Frequency of administration

I take 50,000 IU in one administration in the morning daily

Period of administration

Continuing: Almost 2 months

### Recovery Following Treatment With Vitamin A

Very soon after taking Vitamin A my neck was much more flexible and the aching largely disappeared. I have much more movement in my neck and the aching has now disappeared.

Big improvement in overall mobility, walking and feeling of better health following the rhizolysis in early March 2019 - I do not know if this all due to the procedure or whether this improvement is due to the Vitamin A. but the big improvement in my neck before the procedure was after taking Vitamin A and continued after I stopped taking the Arcoxia.

Examples 5 and 6 below describe the effect of treatment of equine tendon injury with vitamin A. Whist not directed to treatment of concussion, these examples provide evidence for the effect of vitamin A in another type of injury involving deposition of scar tissue.

### EXAMPLE 5

### Tendon Iniurv - Recovery Following Treatment With Vitamin A

| | |
|---|---|
| Patient | Horse subject 1 (polo pony) |
| Age | 14 years |
| Sex | Mare |
| Health status | Good |

### Injury

| | |
|---|---|
| Type of injury | Tendon injury |
| When the injury occurred | 14 Feb. 2019 |
| Cause of the injury | Hyperextension and/or blunt trauma |
| Tissue(s) affected | Tendon |
| Impairments resulting from the injury | Lameness and loss of athletic function |

### Treatment With Conventional Methods

Single dose 30 mg dexamethasone IV to treat the initial acute inflammation followed by 1 g phenylbutazone PO BID for 5 days. Ice applied to affected area for 15 minutes twice a day for 5 days.

### Extent of Recovery Following Treatment With Conventional Methods

Recovery has progressed as expected with these types of lesions in horses. Moderate lameness, heat and pain response to firm digital palpation improved rapidly over the first 7 to 10 days. The horse then started a programme of incremental walking exercise.

### Treatment With Vitamin A

How soon after the injury was treatment was treatment with Vitamin A initiated?

Treatment with Vitamin A was initiated approximately 20 days after the initial injury.

Type of Vitamin A administered

Retinyl palmitate (93.8% Retinol Equivalent)

Route of administration

Oral

Dose of Vitamin A per administration

750,000 IU

Frequency of administration

Twice daily (1,500,000 IU per day)

Period of administration

Continuing (at least 4 weeks)

### Recovery Following Treatment With Vitamin A

Functional recovery has been good considering the severity of the lesion. Ultrasonography has confirmed good reduction in the size of the lesion (notable reduction in size of anechoic pockets of interstitial haemorrhage). Horses are prey species and often do not show signs of overt pain or lameness even with severe lesions. It is not uncommon for such lesions to take between 12 to 18 months to heal.

### EXAMPLE 6 - TREATMENT OF EQUINE TENDON INJURY

This example describes the effect of a pharmaceutical composition comprising vitamin A in treating tendon injury in horses.

Tendon injuries result in the formation of a fibrovascular scar that never attains the characteristics of normal tendon. Tendon healing is characterised by the formation of fibrovascular scar tissue, as tendon has very little intrinsic regenerative capacity. The molecular mechanisms resulting in scar tissue formation after tendon injuries are not well understood (as reviewed in Schneider et al. Rescue plan for Achilles: Therapeutics steering the fate and functions of stem cells in tendon wound healing; Advanced Drug Delivery Reviews 129 2018 352-375). Briefly, in the first few days after injury a blood clot forms that serves as a preliminary scaffold for invading cells followed by a more robust vascular network which is essential for the survival of tenocytes engaged in the synthesis of new fibrous tissue. Thereafter, fibroblasts are recruited to the injured site and produce initially disorganised extracellular matrix components. Following this, a remodelling stage commences characterised by tissue changes resulting in a more fibrous appearance and eventually a scar-like tendon tissue can be observed.

Current biologic treatment strategies have not achieved tendon regeneration but include the use of extracellular matrix patches to provide a scaffold for new cell growth and differentiation (as reviewed in Galatz et al. Tendon Regeneration and Scar Formation: The Concept of Scarless Healing, J. Orthop. Res. 2015, 33(6) 823-831). Platelet rich plasma which comprises a multitude of growth factors normally involved in repair processes has also been investigated for use in tendon repair. However, there is no evidence that either strategy induces tendon regeneration. Tendon injuries are also a particular problem in horses.

Tendon injury has a similar pathophysiology to concussion in that both may be characterised by excessive deposition of scar tissue. Evidence for an effective treatment of tendon injury (including evidence for inhibition of scar tissue formation following tendon injury) is considered to provide evidence for an effective treatment of concussion, for example, through inhibition of scar tissue formation.

### Pharmaceutical Composition Used

Vitamin A palmitate (also known as preformed vitamin A, or retinyl palmitate) mixed with coconut oil to provide a final vitamin A concentration of 10,000 IU/ml.

### Administration of Pharmaceutical Composition

Horses with tendon injury were orally administered vitamin A palmitate mixed with coconut oil, at a dose of 160,000 IU once per day for 14 days.

### Results

Ultrasonographs of the lesions before administration of any pharmaceutical composition comprising vitamin A, and after daily administration of the composition for 14 days are shown for two different horses in FIGS. 2 and 3 (FIG. 2: horse ID 111112; FIG. 3: horse ID REG6).

FIG. 2(a) (before any administration of the composition) shows a subtle core lesion in the lateral aspect of the superficial digital flexor tendon (SDFT) with generalised surrounding tendonitis. FIG. 2(b) (after daily administration of the composition for 14 days) shows that the core lesion has filled in somewhat and is less hypoechoic, suggesting that something has “plugged” the hole. Whilst the nature and quality of the tissue in the lesion is hard to assess with ultrasonography, it certainly appears to be making positive progress after only two weeks.

FIG. 3(a) (before any administration of the composition) shows a nasty SDFT core lesion in the medial aspect not quite involving paratenon. Again the lesion appears to be less hypoechoic on second scan (FIG. 3(b) - after daily administration of the composition for 14 days) suggesting the lesion is filling in with tissue of some sort. Again, the nature and quality of the tissue filling this lesion is hard to assess with ultrasound, but the lesion appears to be making positive progress after only two weeks.

### Conclusions:

2 weeks in the field of equine chronic tendon/ligament injuries is a very short timescale and it is rare to see any significant change in these slow healing structures over such a short time period. In more acute injuries, there is a lot more early activity and ultrasonographic evidence of healing as the tendon responds to injury and the inflammatory cascade process commences.

The results presented here appear to show that vitamin A supplementation (by daily administration of the pharmaceutical composition) has had a positive effect on healing of the lesions after only 2 weeks.

1. Vitamin A for use in the treatment of acute or chronic traumatic brain injury (TBI) in a subject.

2. Use of vitamin A in the manufacture of a medicament for the treatment of acute or chronic TBI in a subject.

3. Vitamin A for use according to claim 1, or use of vitamin A according to claim 2, wherein the acute or chronic TBI is concussion.

4. Vitamin A for use according to claim 1, or use of vitamin A according to claim 2, wherein the acute or chronic TBI is post-concussion syndrome (PCS).

5. Vitamin A for use according to claim 1, or use of vitamin A according to claim 2, wherein the chronic TBI is chronic traumatic encephalopathy (CTE).

6. Vitamin A for use in the prevention, treatment, or amelioration of a brain disorder with delayed onset following a TBI in a subject.

7. Use of vitamin A in the manufacture of a medicament for the prevention, treatment, or amelioration of a brain disorder with delayed onset following a TBI in a subject.

8. Vitamin A for use according to claim 6, or use of vitamin A according to claim 7, wherein the brain disorder with delayed onset is CTE, depression, Parkinson’s disease, dementia, or an anxiety disorder.

9. Vitamin A for use, or use according to any preceding claim, wherein the vitamin A inhibits formation of glial scar tissue in the brain of the subject.

10. Vitamin A for use, or use of vitamin A, according to any preceding claim wherein the subject is a human subject.

11. Vitamin A for use, or use according to any preceding claim, wherein the TBI was sustained when the subject was playing a sport.

12. Vitamin A for use, or use according to any preceding claim, wherein the subject is an athlete, or was an athlete when the TBI was sustained.

13. Vitamin A for use, or use of vitamin A, according to any preceding claim, wherein the vitamin A comprises isolated vitamin A.

14. Vitamin A for use, or use of vitamin A, according to any preceding claim, wherein the vitamin A comprises a preformed vitamin A, such as a retinyl ester or retinol.

15. Vitamin A for use, or use of vitamin A, according to any preceding claim wherein the vitamin A comprises a provitamin A, such as a carotenoid.

16. Vitamin A for use, or use of vitamin A, according to any preceding claim wherein the vitamin A comprises a bioactive form of vitamin A, such as retinal or retinoic acid.

17. Vitamin A for use, or use of vitamin A, according to any preceding claim for administration at a dose in excess of a Tolerable Upper Limit Intake Level (UL) for the subject.

18. Vitamin A for use, or use of vitamin A, according to claim 17 for administration to the subject at a dose of >10,000 to 100,000 IU vitamin A per day.

19. Vitamin A for use, or use of vitamin A, according to claim 18 for administration to the subject at a dose of about 25,000-50,000, 25,000-75,000, 25,000-100,000, 50,000-100,000, or 75,000-100,000 IU vitamin A per day.

20. Vitamin A for use, or use of vitamin A, according to any preceding claim for administration to the subject once per day.

21. Vitamin A for use, or use of vitamin A, according to claim 20 for administration for at least 3 days from the day of first administration to the subject.

22. Vitamin A for use, or use of vitamin A, according to claim 20 for administration for at least a week, at least a month, or at least 6 months from the day of first administration to the subject.

23. Vitamin A for use, or use of vitamin A, according to any of claims 20 to 22 for administration to the subject for up to 6 years from the day of first administration to the subject.

24. Vitamin A for use, or use of vitamin A, according to any preceding claim for administration systemically.

25. Vitamin A for use, or use of vitamin A, according to claim 24 for administration to the subject orally or intravenously.

26. A method of treating acute or chronic TBI in a subject, which comprises administering to the subject an effective amount of vitamin A.

27. A method according to claim 26, wherein the acute or chronic TBI is concussion.

28. A method according to claim 26, wherein the acute or chronic TBI is post-concussion syndrome (PCS).

29. A method according to claim 26, wherein the chronic TBI is chronic traumatic encephalopathy (CTE).

30. A method of preventing, treating, or ameliorating a brain disorder with delayed onset following a TBI in a subject, which comprises administering to the subject an effective amount of vitamin A.

31. A method according to claim 30, wherein the brain disorder with delayed onset is CTE, depression, Parkinson’s disease, dementia, or an anxiety disorder.

32. A method according to any of claims 26 to 31, wherein the vitamin A inhibits formation of glial scar tissue in the brain of the subject.

33. A method according to any of claims 26 to 32, wherein the subject is a human subject.

34. A method according to any of claims 26 to 33, wherein the TBI was sustained when the subject was playing a sport.

35. A method according to any of claims 26 to 34, wherein the subject is an athlete, or was an athlete when the TBI was sustained.

36. A method according to any of claims 26 to 35, wherein the vitamin A comprises isolated vitamin A.

37. A method according to any of claims 26 to 36, wherein the vitamin A comprises a preformed vitamin A, such as a retinyl ester or retinol.

38. A method according to any of claims 26 to 37, wherein the vitamin A comprises a provitamin A, such as a carotenoid.

39. A method according to any of claims 26 to 38, wherein the vitamin A comprises a bioactive form of vitamin A, such as retinal or retinoic acid.

40. A method according to any of claims 26 to 39, wherein the vitamin A is administered at a dose in excess of a Tolerable Upper Limit Intake Level (UL) for the subject.

41. A method according to claim 40, wherein the vitamin A is administered to the subject at a dose of >10,000 to 100,000 IU vitamin A per day.

42. A method according to claim 41, wherein the vitamin A is administered to the subject at a dose of about 25,000-50,000, 25,000-75,000, 25,000-100,000, 50,000-100,000, or 75,000-100,000 IU vitamin A per day.

43. A method according to any of claims 26 to 42, wherein the vitamin A is administered to the subject once per day.

44. A method according to claim 43, wherein the vitamin A is administered to the subject once per day for at least 3 days from the day of first administration to the subject.

45. A method according to claim 43, wherein the vitamin A is administered to the subject once per day for at least a week, at least a month, or at least 6 months from the day of first administration to the subject.

46. A method according to any of claims 43 to 45, wherein the vitamin A is administered to the subject for up to 6 years from the day of first administration to the subject.

47. A method according to any of claims 26 to 46, wherein the vitamin A is administered systemically to the subject.

48. A method according to claim 47, wherein the vitamin A is administered orally or intravenously to the subject.

49. A method according to any of claims 26 to 48, wherein the vitamin A is first administered to the subject within a week of the TBI.

50. A method according to any of claims 26 to 49, wherein the vitamin A is first administered to the subject within a day of the TBI.
